(19) United States
(12) Patent Application Publication
(10) Pub. No.: US 2006/0242753 A1
(43) Pub. Date: Nov. 2, 2006

(54) Swim mask

(75) Inventor: Chih-Cheng Shiue, Escondido, CA (US)

Correspondence Address: BRUCE H. TROXELL, SUITE 1404, 5205 LEESBURG PIKE, FALLS CHURCH, VA, 22041, US

(73) Assignee: QDS Injection Molding LLC

(21) Appl. No.: 11/117,362

(22) Filed: Apr. 29, 2005

(51) Int. Cl. A61F 9/02

(52) U.S. Cl. 2/447

(57) ABSTRACT

Provided is a swim mask having a member to cover at least the nose. The swim mask further comprises a peripheral skirt formed of elastomeric material. A portion of the skirt between the lenses is thus flexible such that a person wears the swim mask will feel comfort on a portion on top of the nose and between the eyes by buffering pressing of the portion of the skirt between the lenses thereon. The invention can eliminate the discomfort caused by pressing a hard member on the portion on top of the nose and between the eyes. Moreover, the invention can decrease a distance between the lens and the eye so as to increase view of the wearer.

### FIELD OF INVENTION

The present invention relates to goggles or masks for swimming, snorkeling, or diving and more particularly to a swim mask having a bridge (i.e., member between two lenses) formed of elastomeric material (e.g., silicone rubber or the like).

### BACKGROUND OF THE INVENTION

Conventionally, coverings for water sports, swimming, snorkeling, or diving can be classified as swim goggles (e.g., ones without members to cover the nose) and swim masks (e.g., ones with members to cover at least the nose and the eyes). For swim goggles, they are designed to enable persons to swim in a swimming pool. For swim masks, they are particularly suitable for swimming, snorkeling, or diving.

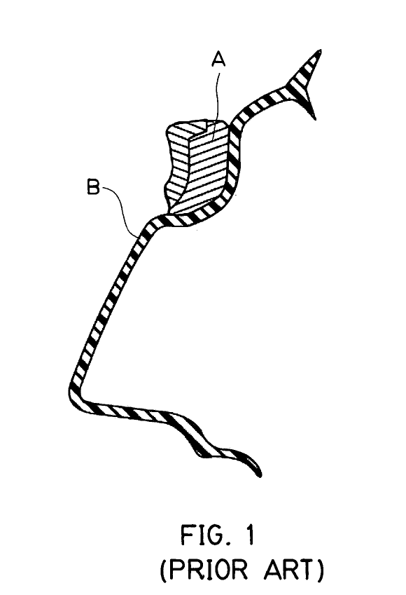

Commercially available swim masks, whether having a single lens or two spaced lenses, have the same drawback which is that a bridge member (i.e., located on top of the nose and between two eyes) of the swim mask is formed of hard material (see frame A or bridge frame in FIG. 1). In use, a slight pressing of the mask will concentrate force on a portion on top of the nose and between the eyes (i.e., bridge) and therearound. Skirt B is pressed by the frame A. The user may feel discomfort because a hard member presses on the bridge. For lessening the discomfort, the manufacturer of the art may add a space in back of the hard member (i.e., a portion on top of the nose and between the eyes) such that the pressing may not exert on the bridge directly. However, it may adversely increase a distance between the lens and the eye, limit view of the swim mask, and decrease the size of the swim mask. For swim masks having a single lens, distance between the lens and the bridge is further decreased. As such, the degree of discomfort due to pressing the hard member on the bridge is further serious. A proposal for eliminating the discomfort is the same as above by adding a space between the hard member and the bridge such that the pressing may not exert on the bridge directly. Hence, a need has arisen for a improved swim mask in order to overcome the drawback of the prior art and contribute significantly to the advancement of the art.

### SUMMARY OF THE INVENTION

It is therefore an object of the present invention to provide a swim mask having a member to cover the nose. The swim mask comprises a peripheral skirt and two lenses fitted in the skirt wherein a portion of the skirt between the lenses is flexible.

It is another object of the present invention to provide a swim mask comprising a peripheral skirt and two lenses wherein the lenses are formed with the skirt by assembling or molding such that no hard member is formed between the lenses. By utilizing this swim mask, pressure generated by wearing the lenses will not completely bear upon a portion on top of the nose and between the eyes because a portion of the skirt between the lenses is flexible. The wearer will not feel discomfort on the portion on top of the nose and between the eyes by the reduced pressure.

The above and other objects of the present invention are realized by providing a swim mask having a flexible member between the lenses such that pressure generated by wearing the lenses and borne upon a portion on top of the nose and between the eyes is much decreased. Moreover, the present invention can be designed and constructed in a way to decrease a distance between the lens and the eye for increasing view of the wearer because there is no hard member between the lenses.

The above and other objects, features and advantages of the present invention will become apparent from the following detailed description taken with the accompanying drawings.

### BRIEF DESCRIPTION OF THE INVENTION

FIG. 1 is a sectional view of a prior art swim mask;

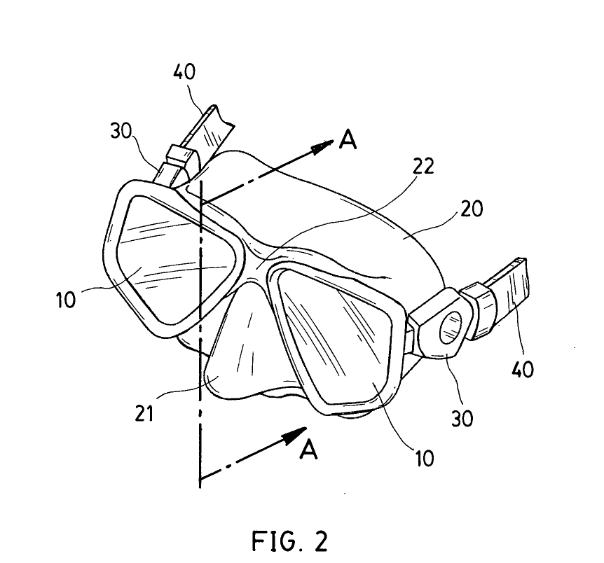

FIG. 2 is a perspective view of a preferred embodiment of swim mask according to the invention;

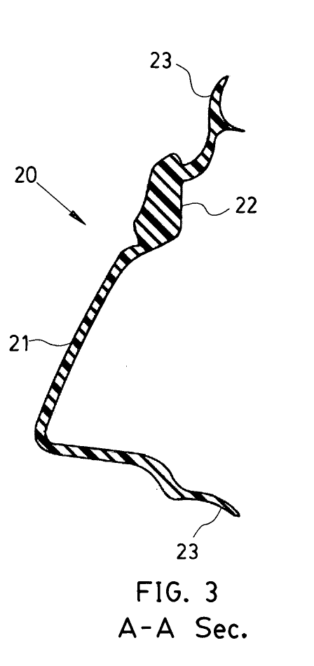

FIG. 3 is a sectional view taken along line A-A of FIG. 2; and

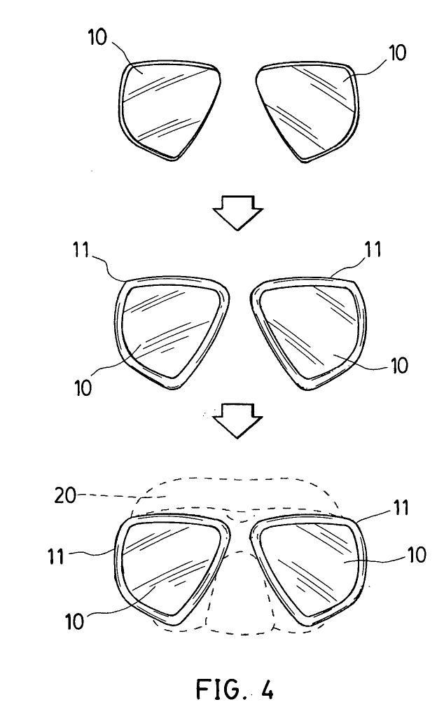

FIG. 4 illustrates three steps of forming the swim mask of the invention.

### DETAILED DESCRIPTION OF THE INVENTION

Referring to FIG. 2, there is shown a swim mask constructed in accordance with a preferred embodiment of the invention. The swim mask comprises two spaced lenses 10, a peripheral skirt 20, a strap 40, and two connecting members 30 each interconnected either end of the strap 40 and an endpiece of either lens 10. The lenses 10 and the skirt 20 are the subjects of the invention as detailed below. However, the connecting members 30 and the strap 40 can be constructed in any manner well understood by those skilled in the art. Accordingly, further description thereof is omitted for purpose of brevity.

Referring to FIGS. 2 and 3, the skirt 20 comprises a nose portion 21, a bridge portion 22, and a peripheral portion 23. The nose portion 21 is adapted to cover the nose of a user. The bridge portion 22 is adapted to interconnect the lenses 10 and is formed of elastomeric material. The peripheral portion 23 is adapted to enable the skirt 20 to closely engage with the face of the user. The skirt 20 is formed of elastomeric material (e.g., silicone rubber, rubber or the like).

Referring to FIG. 4, a manufacturing process of the swim mask of the invention is detailed below. In step 1, two separate lenses 10 are prepared. In step 2, a frame 11 made of elastomeric material or hard material is formed around either lens 10. Alternatively, the flexible frames 11 are formed around the lenses 10 by assembling or snapping. In the final step 3, form the swim mask by molding (or assembling or gluing) the lenses 10 and the frames 11 in the skirt 20.

Note that in an alternative manufacturing process of the swim mask, the lenses 10 and the skirt 20 are formed separately. Finally, snap the lenses 10 in two predetermined lens openings of the skirt 20. Advantageously, structure between the lenses 10 (i.e., the bridge) is still flexible.

While the invention herein disclosed has been described by means of specific embodiments, numerous modifications and variations could be made thereto by those skilled in the art without departing from the scope and spirit of the invention set forth in the claims.

1-3. (canceled)

4. A swim mask comprising:
a) a peripheral shirt made of an elastomeric material and having:
i) two lens openings;
ii) a bridge portion located between the two lens openings;
iii) a peripheral portion surrounding the two lens openings; and
iv) a nose portion located between the two lens openings and below the bridge portion;
b) two lenses, one of the two lenses is connected to the each of the two lens openings;
c) a strap; and
d) two connecting members, a first end of one of the two connecting members is connected to each of two opposing ends of the strap and a second end of one of the two connecting members is connected to an end piece of each of the two lenses,
wherein the bridge portion is flexible and has a thickness greater than a thickness of the peripheral portion and the nose portion.

5. The swim mask according to claim 4, wherein one of the two lenses is connected to the each of the two lens openings by snapping.

6. The swim mask according to claim 4, further comprising a frame formed around each of the two lenses.

7. The swim mask according to claim 4, wherein the nose portion covers an entire surface of a nose selectively sealing the nose therein.
